(19) United States
(12) Patent Application Publication
(10) Pub. No.: US 2013/0317293 A1
(43) Pub. Date: Nov. 28, 2013

(54) DEVICES, METHODS, AND SYSTEMS FOR DELIVERING IMPLANTS TO A LUNG REGION

(71) Applicant: PulmonX Corporation, Redwood City, CA (US)

(72) Inventors: Ryan Olivera, Granite Bay, CA (US); Hoang NGUYEN, San Jose, CA (US)

(73) Assignee: PulmonX Corporation, Redwood City, CA (US)

(21) Appl. No.: 13/900,082

(22) Filed: May 22, 2013

Related U.S. Application Data
(60) Provisional application No. 61/650,357, filed on May 22, 2012.

(51) Int. Cl. A61B 1/018; A61B 1/267; A61M 25/01

(52) U.S. Cl. CPC A61B 1/018; A61M 25/01; A61B 1/2676
USPC 600/104

(57) ABSTRACT

Devices, methods, and systems for accessing a lung region are provided. The system includes a delivery catheter having a proximal section, a distal section, and a body disposed in-between, wherein the distal region comprises at least one curved section; and a bronchoscope having a proximal section, a distal section, and a body disposed in-between, wherein the bronchoscope is configured to receive the delivery catheter; wherein the delivery catheter is configured to affect a curvature of the distal section of the bronchoscope when the distal section of the delivery catheter is advanced at least partially through the distal end of the bronchoscope.

### CROSS-REFERENCES TO RELATED APPLICATIONS

This application claims priority of U.S. Provisional Patent Application Ser. No. 61/650,357 entitled “Bronchial Delivery System”, filed May 22, 2012, the disclosure of which is incorporated by reference in its entirety.

### FIELD OF THE INVENTION

Present embodiments relate generally to devices, methods, and systems for accessing and delivering one or more implants to a lung region.

### BACKGROUND OF THE INVENTION

Pulmonary diseases, such as chronic obstructive pulmonary disease, (COPD), reduce the ability of one or both lungs to fully expel air during the exhalation phase of the breathing cycle. Such diseases are accompanied by chronic or recurrent obstruction to air flow within the lung. Because of the increase in environmental pollutants, cigarette smoking, and other noxious exposures, the incidence of COPD has increased dramatically in the last few decades and now ranks as a major cause of activity-restricting or bed-confining disability in the United States. COPD can include such disorders as chronic bronchitis, bronchiectasis, asthma, and emphysema.

It is known that emphysema and other pulmonary diseases reduce the ability of one or both lungs to fully expel air during the exhalation phase of the breathing cycle. One of the effects of such diseases is that the diseased lung tissue is less elastic than healthy lung tissue, which is one factor that prevents full exhalation of air. During breathing, the diseased portion of the lung does not fully recoil due to the diseased (e.g., emphysematic) lung tissue being less elastic than healthy tissue. Consequently, the diseased lung tissue exerts a relatively low driving force, which results in the diseased lung expelling less air volume than a healthy lung. The reduced air volume exerts less force on the airway, which allows the airway to close before all air has been expelled, another factor that prevents full exhalation.

The problem is further compounded by the diseased, less elastic tissue that surrounds the very narrow airways that lead to the alveoli, which are the air sacs where oxygen-carbon dioxide exchange occurs. The diseased tissue has less tone than healthy tissue and is typically unable to maintain the narrow airways open until the end of the exhalation cycle. This traps air in the lungs and exacerbates the already-inefficient breathing cycle. The trapped air causes the tissue to become hyper-expanded and no longer able to effect efficient oxygen-carbon dioxide exchange.

In addition, hyper-expanded, diseased lung tissue occupies more of the pleural space than healthy lung tissue. In most cases, a portion of the lung is diseased while the remaining part is relatively healthy and, therefore, still able to efficiently carry out oxygen exchange. By taking up more of the pleural space, the hyper-expanded lung tissue reduces the amount of space available to accommodate the healthy, functioning lung tissue. As a result, the hyper-expanded lung tissue causes inefficient breathing due to its own reduced functionality and because it adversely affects the functionality of adjacent healthy tissue.

Some recent treatments include the use of devices that isolate a diseased region of the lung in order to reduce the volume of the diseased region, such as by collapsing the diseased lung region. According to such treatments, a delivery catheter is used to implant one or more implantable devices in airways feeding a diseased region of the lung to regulate fluid flow to the diseased lung region in order to fluidly isolate the region of the lung. These implanted implantable devices can be, for example, one-way valves that allow flow in the exhalation direction only, occluders or plugs that prevent flow in either direction, or two-way valves that control flow in both directions.

Various treatments typically require the ability to access a lung region to diagnose the diseased region and to deliver a device to the diseased region. Thus, there is a need for improved access devices to facilitate access.

### SUMMARY OF THE INVENTION

Present disclosure relates to aspects of devices, methods, and systems for accessing and delivering one or more implants a lung region.

In one aspect, a system for accessing a lung region comprises a delivery catheter with a proximal section, a distal section, and a body disposed in-between, wherein the distal section of the delivery catheter comprises at least one curved section, and a bronchoscope with a proximal section, a distal section, and a body disposed in-between, wherein the bronchoscope is configured to receive the delivery catheter and the delivery catheter is configured to affect a curvature of the distal section of the bronchoscope when the distal section of the delivery catheter is advanced at least partially through the distal section of the bronchoscope.

In another aspect, a bronchial delivery catheter comprises a distal section, a proximal section, and a body disposed in-between, wherein the distal section comprises a housing section that is configured to house one or more bronchial implants and a first curved section disposed in-between the housing section and the body.

This and other aspects of the present disclosure are described herein.

### BRIEF DESCRIPTION OF THE DRAWINGS

Present embodiments have other advantages and features which will be more readily apparent from the following detailed description and the appended claims, when taken in conjunction with the accompanying drawings, in which:

FIG. 1 shows one embodiment of a delivery catheter comprising a distal section with a curved section;

FIG. 2 shows an anterior view of a pair of human lungs and a bronchial tree with a bronchial access system comprising a bronchoscope that is inserted within the bronchial passageway;

FIG. 3a shows a perspective view of a bronchoscope;

FIG. 3b shows an enlarged view of a distal section of a bronchoscope;

FIG. 4a shows a bronchoscope with a delivery catheter inserted therein;

FIG. 4b shows a bronchoscope with a delivery catheter inserted therein, wherein a curvature of a section of the bronchoscope is affected by the delivery catheter;

FIG. 4c shows a bronchoscope placed at a junction of a branched bronchial passageway;

FIG. 4d shows a bronchoscope placed at a junction of a branched bronchial passageway with a delivery catheter inserted therein to change the orientation of the distal section of the bronchoscope;

FIG. 5 shows a delivery catheter with an inner section and an outer section;

FIGS. 6a-6c show a delivery catheter where the inner section is advanced to affect the outer section.

### DETAILED DESCRIPTION

Although the detailed description contains many specifics, these should not be construed as limiting the scope of the disclosure but merely as illustrating different examples and aspects of the disclosure. It should be appreciated that the scope of the disclosure includes other embodiments not discussed herein. Various other modifications, changes and variations which will be apparent to those skilled in the art may be made in the arrangement, operation and details of the method, device, and system of the present embodiments disclosed herein without departing from the spirit and scope of the disclosure as described here.

Throughout the specification and claims, the following terms take the meanings explicitly associated herein unless the context clearly dictates otherwise. The meaning of “a”, “an”, and “the” include plural references. The meaning of “in” includes “in” and “on.” Referring to the drawings, like numbers indicate like parts throughout the views. Additionally, a reference to the singular includes a reference to the plural unless otherwise stated or inconsistent with the disclosure herein.

The word “exemplary” is used herein to mean “serving as an example, instance, or illustration.” Any implementation described herein as “exemplary” is not necessarily to be construed as advantageous over other implementations.

Throughout this disclosure, reference is made to the term “implantable device”. As used herein, the term “implantable device” refers to various collapsible self-expanding implants including implants configured as various flow control devices and pulmonary implants capable of being placed within a lung region to treat pulmonary disorders. Furthermore, the implantable device may be a device to treat vascular, urinary, biliary, esophageal, and renal tracts and the like.

Throughout this disclosure, reference is made to the term “lung region”. As used herein, the term “lung region” refers to a defined division or portion of a lung. For purposes of example, lung regions are described herein with reference to human lungs, wherein some exemplary lung regions include lung lobes and lung segments. Thus, the term “lung region” as used herein can refer, for example, to a lung lobe or a lung segment. Such nomenclature conforms to nomenclature for portions of the lungs that are known to those skilled in the art. However, it should be appreciated that the term “lung region” does not necessarily refer to a lung lobe or a lung segment, but can refer to some other defined division or portion of a human or non-human lung.

In one embodiment of bronchial implantable device delivery operation, a bronchial implantable device is delivered to a target location in a lung region by mounting the bronchial implantable device in a housing at the distal end of a delivery catheter and then inserting the delivery catheter into the bronchial passageway. Once the housing is positioned at a target location in the bronchial passageway, the bronchial isolation device is ejected from the housing and deployed within the passageway. The delivery catheter may be inserted into the patient's mouth or nose, through the trachea, and down to a target location in the bronchial passageway.

The following references describe exemplary delivery devices: U.S. Pat. No. 7,717,115 entitled “Delivery Methods and Devices for Implantable Bronchial Isolation Devices”; U.S. Pat. No. 5,954,766 entitled “Body Fluid Flow Control Device”; U.S. patent application Ser. No. 09/797,910, entitled “Methods and Devices for Use in Performing Pulmonary Procedures”; U.S. patent application Ser. No. 10/270,792, entitled “Bronchial Flow Control Devices and Methods of Use”; and U.S. patent application Ser. No. 10/448,154, entitled “Guidewire Delivery of Implantable Bronchial Isolation Devices in Accordance with Lung Treatment”. The foregoing references are all incorporated by reference in their entirety and are all assigned to the assignee of the instant application.

Various catheter characteristics may influence torqueability, pushability, and/or maneuverability and the ease of arriving at the desired lung region. For example, a high degree of rigidity of a delivery catheter may improve the torqueability and pushability of the delivery catheter; however, a high degree of rigidity may hinder maneuverability and placement especially in tortuous anatomical regions. The present disclosure describes various devices and methods for delivering one or more bronchial implantable devices to a location in a lung region such as a bronchial passageway. Specifically, the present disclosure describes embodiments of a delivery catheter configured to increase maneuverability, particularly through anatomical bends and to facilitate placement of the implantable device within the desired lung region.

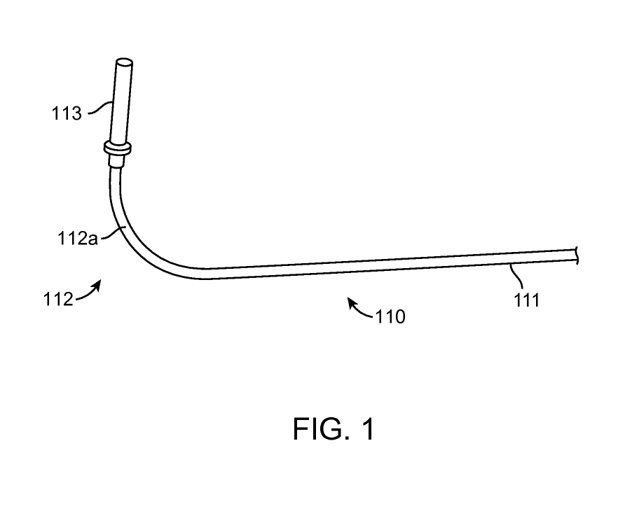

Referring now to FIG. 1, where in one embodiment, the delivery catheter 110 comprises a catheter body section 111, a distal section 112, and a housing section 113 configured to house one or more implants. The housing section 113 can be manufactured of a rigid material, such as steel. The housing section 113 can also be flexible or collapsible. Although the housing section 113 is shown having a cylindrical shape, it should be appreciated that the housing section 113 can have other shapes that are configured to receive the implantable device such as a bronchial isolation device therein.

The body section 111 is disposed between the distal section 112 and the proximal section (not shown). The distal section 112 is configured to comprise at least one curved section 112a that assumes a curvature wherein the housing section 113 connected to the curved section 112a is directed at an angle with respect to the body section 111.

The curved section 112a may be configured with various curvatures such that the housing section 113 is oriented at various angles relative to the body section 111. As seen in FIG. 1, the curved section 112a may assume a curvature configuration where the housing section 113, in one embodiment, the distal section 112 and at least a section of the body section 111 assumes a “J” shaped configuration.

In one embodiment, the curved section 112a comprises a first material located generally along an outer surface on the outer radius of the curved section 112a and a second material located generally along an inner surface on the inner radius of the curved section 112a.

In one embodiment, the first material has a greater resistance to bending stress or torque than the more elastic second material. In such embodiment, locating of the first material along the outer surface of the delivery catheter 110 reinforces the curved section 112a so that it is better able to generally retain its shape even when subjected to a force during engagement. In an alternate embodiment, the locations of the first and second materials may be reversed.

It is further contemplated that the distal section 112 may comprise multiple curved sections such that the distal section 112 assumes multiple discrete angles. In one embodiment, the multiple curved sections may allow the distal section 112 to assume a multi-planar curvature configuration. The delivery catheter 110 comprising one or more curved sections may facilitate access to branching bronchial passageways, wherein the curved section may aid or allow the housing section to align with a branching bronchial passageway.

Additionally and optionally, it is contemplated that in one embodiment, the distal section 112 of the delivery catheter 110 is configured to be more flexible than the body section 111. The flexible distal section 112 enables greater maneuverability whereas the stiffer body section 111 maintains torqueability and/or pushability.

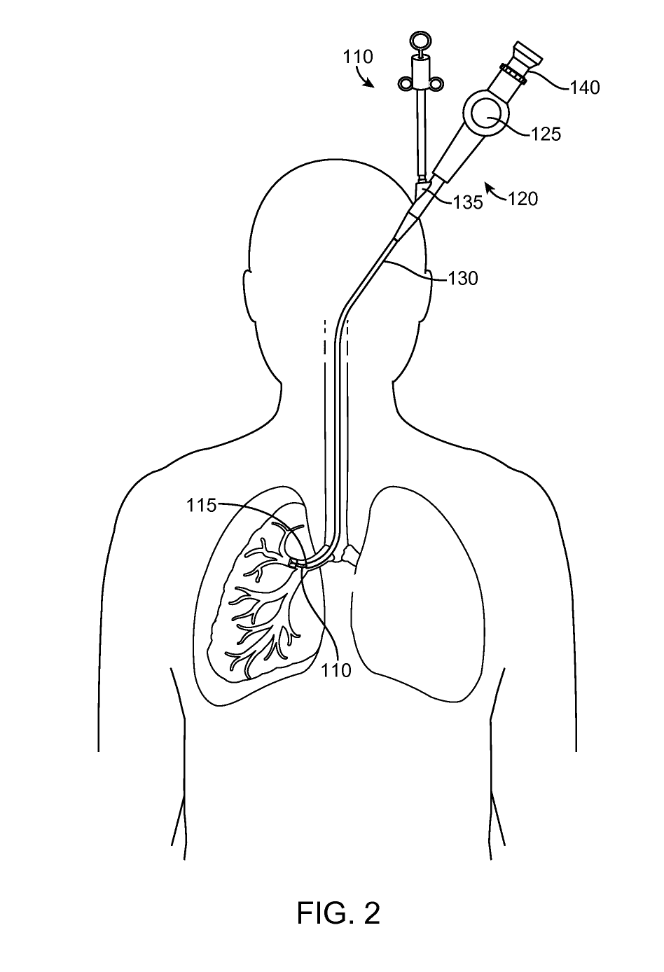

Another aspect of the present embodiments relate to a bronchial accessing system comprising a delivery catheter 110 with at least one curved section 112a and a bronchoscope 120. As described above, the bronchial implantable device is delivered to a target location in a lung region by mounting the bronchial implantable device in a housing section 113 at the distal section of a delivery catheter 110 and then inserting the delivery catheter 110 into the bronchial passageway of a lung region. In one embodiment, the delivery catheter 110 may be inserted directly into the trachea and the bronchial passageway. In another embodiment, as shown in FIG. 2, a bronchoscope 120 assists in the insertion of the delivery catheter 110 through the trachea and into the bronchial passageway. The method that uses the bronchoscope 120 is referred to as the “transcopic” method. According to the transcopic method, the delivery catheter 110 is inserted into the working channel of the bronchoscope 120, which is deployed to the bronchial passageway either before or after the delivery catheter has been inserted into the bronchoscope 120.

An exemplary embodiment of the bronchoscope 120 is shown in FIG. 2. The bronchoscope 120 has a steering mechanism 125, a delivery shaft 130, a working channel entry port 135, and a visualization eyepiece 140. FIG. 2 shows the bronchoscope 120 positioned with its distal end at the right primary bronchus. The delivery catheter 110 is positioned within the bronchoscope 120 such that the delivery catheter's distal end and the attached bronchial isolation device 115 protrude outward from the distal end of the bronchoscope 120, as shown in FIG. 2.

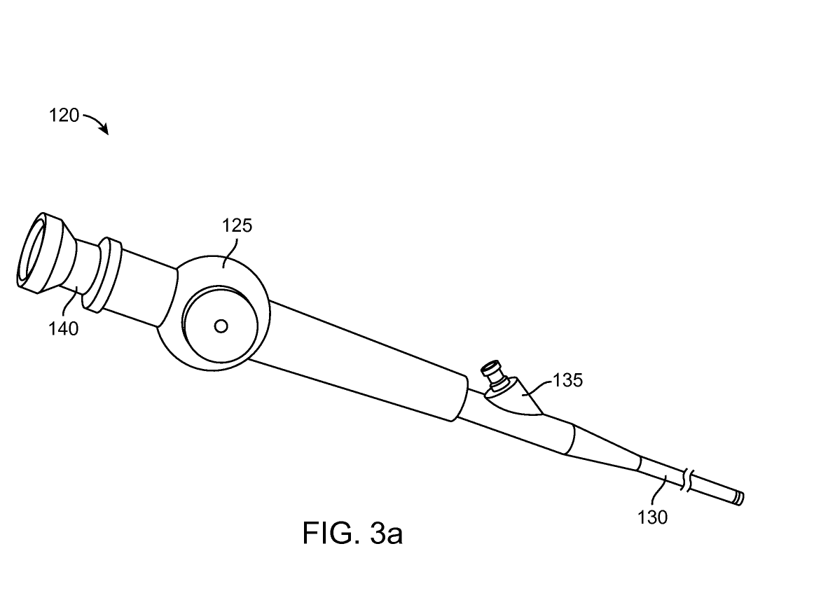

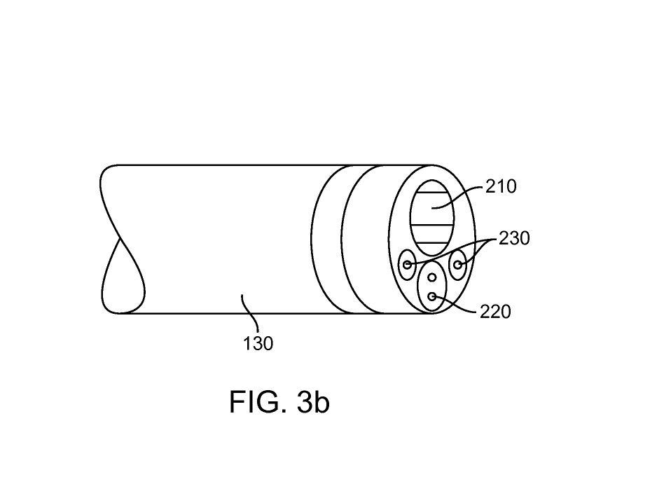

FIG. 3a shows an enlarged view of the bronchoscope 120, including the steering mechanism 125, delivery shaft 130, working channel entry port 135, and visualization eyepiece 140. In addition, the bronchoscope 120 can also include a fiber optic bundle mounted inside the length of the bronchoscope 120 for transferring an image from the distal end to the eyepiece 140. In one embodiment, the bronchoscope 120 also includes a camera or charge-coupled device (CCD) for generating an image of the bronchial tree. FIG. 3b shows an enlarged view of the distal section of the bronchoscope 120. A working channel 210 (sometimes referred to as a biopsy channel and as shown in FIG. 3b) extends through the delivery shaft 130 and communicates with the entry port 135 (shown in FIG. 3a) at the proximal end of the bronchoscope 120. The working channel 210 can sometimes be formed by an extruded plastic tube inside the body of the bronchoscope 120. The bronchoscope 120 can also include various other channels, such as a visualization channel 220 that communicates with the eyepiece 140 and one or more illumination channels 230. It should be appreciated that the bronchoscope can have a variety of configurations and is not limited to the embodiment shown in the figures. For example, in an alternative embodiment, the working channel 210 may be formed of a flexible material and temporarily or permanently attached to the outside of the delivery shaft 130.

A distal section of the bronchoscope 120 may be configured with a pre-set degree of a curvature to ease access and maneuverability. However, the distal section may lose at least a degree of the pre-set curvature during insertion and when it traverses through the trachea and bronchial passageway. By using a delivery catheter 110 with at least one curved section 112a such as the embodiment of the delivery catheter shown in FIG. 1, wherein the curved distal section 112a is placed at least partially through the distal section of the bronchoscope 120, the curvature of the bronchoscope 120 may be maintained, affected, transformed, or amplified. In one embodiment, maintaining, transforming, or amplifying the curvature of the distal section of the bronchoscope 120 allows or facilitates the bronchoscope 120 to traverse anatomical bends and to improve the accessibility of the branched bronchial passageways by allowing the alignment of the bronchoscope 120 with the opening of a branched bronchial passageway. Furthermore, by affecting the curvature of the distal section of the bronchoscope, it may improve visualization of the lung region by directing the distal section of the bronchoscope towards a region of interest in the lung region. Additionally, it is further contemplated that embodiments of the delivery catheter 110 may be configured to maintain the curvature of the distal section of the bronchoscope 120 by fortifying the rigidity of at least a section of the distal section of the bronchoscope 120.

Specifically, the delivery catheter 110 may be used to affect or transform the curvature of at least a section of the distal section of the bronchoscope 120 to assume a new configuration as a result of the interactions between at least a section of the bronchoscope 120 and the delivery catheter 110. The degree of transformation to the curvature of the distal section of the bronchoscope 120 may be calibrated by the differences between one or more curvatures of at least one section of the distal end of the bronchoscope 120, the rigidity, elasticity, shape memory properties, and/or other material properties of the bronchoscope and the delivery catheter 110, the difference in the material properties between the bronchoscope and the delivery catheter, or any combination thereof.

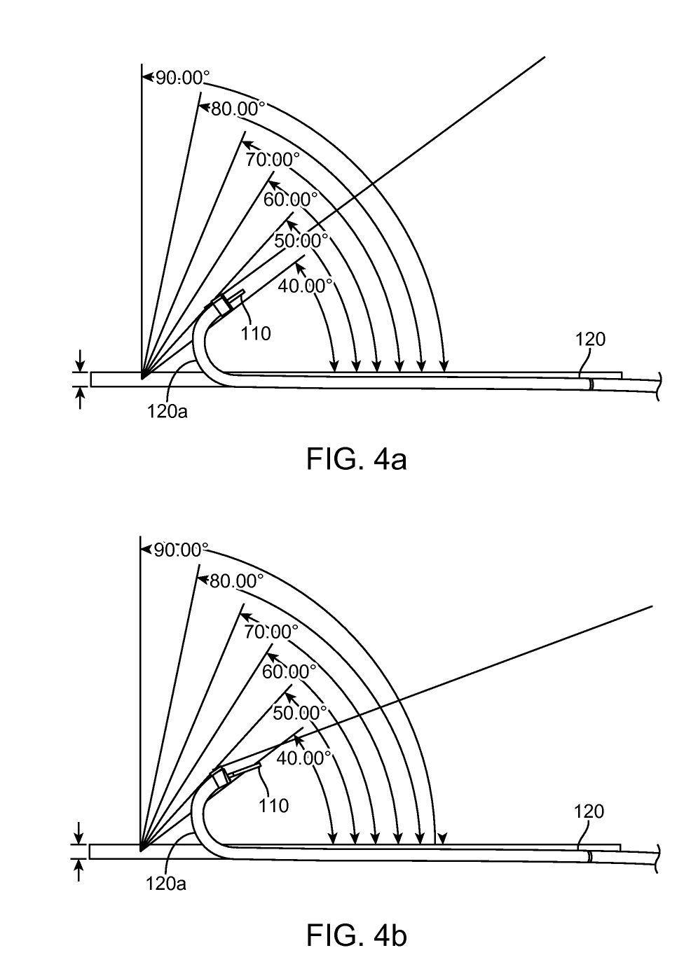

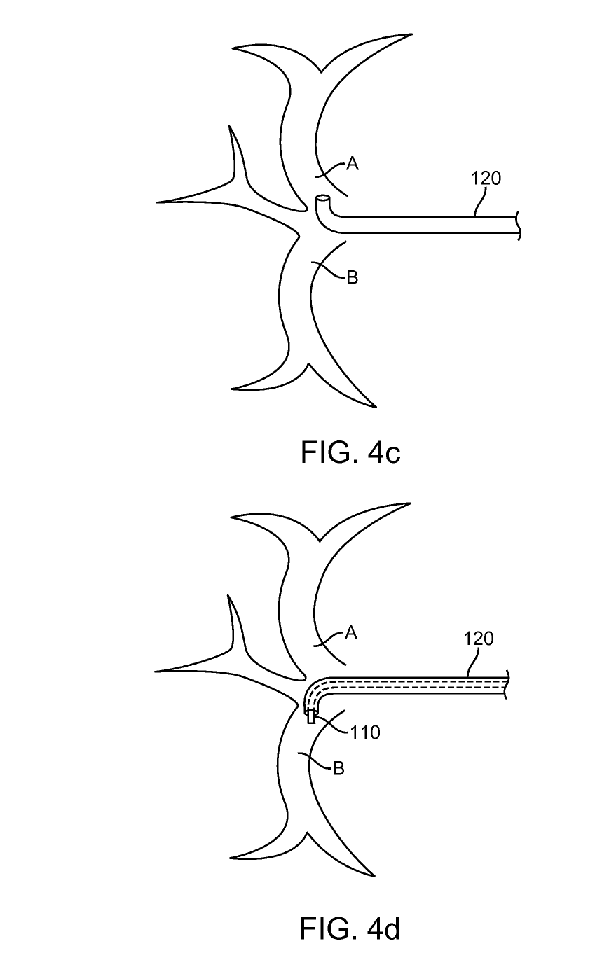

Referring now to FIGS. 4a-4b, where embodiments of the bronchial accessing system are exemplarily shown. As seen in FIGS. 4a-4b, the bronchial accessing system comprises a bronchoscope with a delivery catheter inserted therein. In FIG. 4a, a bronchoscope 120 is shown with a delivery catheter 110 inserted therein. When the delivery catheter 110 is advanced further into the bronchoscope 120, as seen in FIG. 4b, the degree of the curvature of a curved section 120a of the bronchoscope 120 is altered as the delivery catheter 110 advances in the distal direction within the bronchoscope 120.

Additionally, the delivery catheter 110 may be configured to alter the orientation of the distal section bronchoscope 120 to achieve better alignment of the bronchoscope 120 with a targeted branched bronchial passageway. Referring now to FIG. 4c, where a bronchoscope 120 placed near a branch junction within the bronchial passageway. As seen in FIG. 4c, the distal end of the bronchoscope 120 is oriented towards the branched bronchial passageway A. In one embodiment, a section of the distal end of the bronchoscope 120 is configured to have greater degree of elasticity than other sections of the bronchoscope 120. Thereafter, as seen in FIG. 4d, a delivery catheter 110 is inserted into the bronchoscope 120. In one embodiment, the delivery catheter 110 is configured with rigidity, elasticity, and shape memory properties and curvature configuration. As the delivery catheter 110 is advanced towards the distal section of the bronchoscope 120, the delivery catheter 110 interacts with the distal end of the bronchoscope 120 such that the orientation of the distal end of the bronchoscope 120 is altered to be substantially aligned with branch bronchial passageway B.

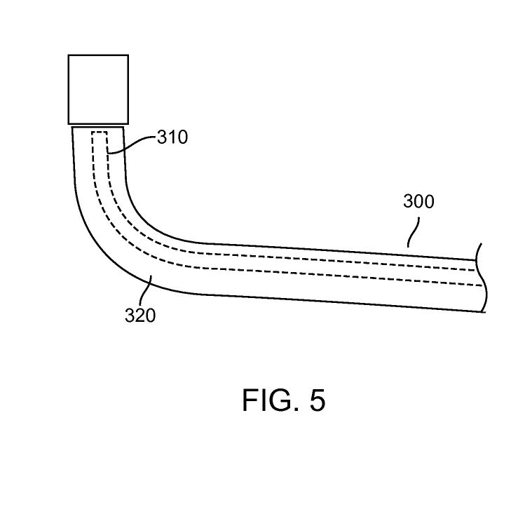

Referring now to FIG. 5, where in one embodiment, a delivery catheter 300 comprises an elongated inner section 310 that is slidably positioned within an outer section 320 that the outer section 320 can slidably move relative to the inner section 310 along the length of the catheter 300.

In one embodiment, the outer section 320 and the inner section 310 may both comprise one or more curved sections. In another embodiment, the inner section 310 or the outer section 320 may be configured to be substantially straight without the any curved section. In one embodiment, the curved sections of the inner section 310 and the other section 320 are configured with substantially similar curvatures. In another embodiment, the curved sections of the inner section 310 and the outer section 320 are configured with different curvatures. For example, in one embodiment, the outer section 320 may be configured with a first pre-set curved section while the inner section 310 is configured with a second preset curved section of different curvature.

The inner section 310 and the outer section 320 may be constructed of different material to create variations to the elasticity, stiffness or rigidity, shape memory properties between the inner section and the outer section. In one embodiment, at least a section of the inner section 310, such as the distal section is constructed of first material preferably has a greater stiffness or rigidity than that of the outer section 320, and hence a greater resistance to bending stress or torque, while at least a section of the outer section 320, such as the distal section, is constructed of an second material of greater elasticity than the inner section 310. In an alternative embodiment, the configuration of the constructed material may be revered or any combination thereof.

Additionally, the inner section 310 of the delivery catheter 300 can include a central guidewire lumen that extends through the entire length of the catheter 300. The central guidewire lumen of the inner section is sized to receive a guidewire, which can be used during deployment of the catheter 300 to guide the catheter 300 to a location in a bronchial passageway.

In another embodiment, an actuation handle (not shown) may be located at the proximal end of the catheter 300. The actuation handle can be actuated to slidably move the outer section 320 in a proximal direction relative to the inner section 310 with the inner section 310 remaining fixed relative to the actuation handle. During such movement, the outer section 320 slides over the inner section 310. Generally, the actuation handle includes a first piece and a second actuation piece, which is moveable relative to the first piece. The outer section 320 of the catheter can be moved relative to the inner section 310 by moving the first piece of the handle relative to the second piece.

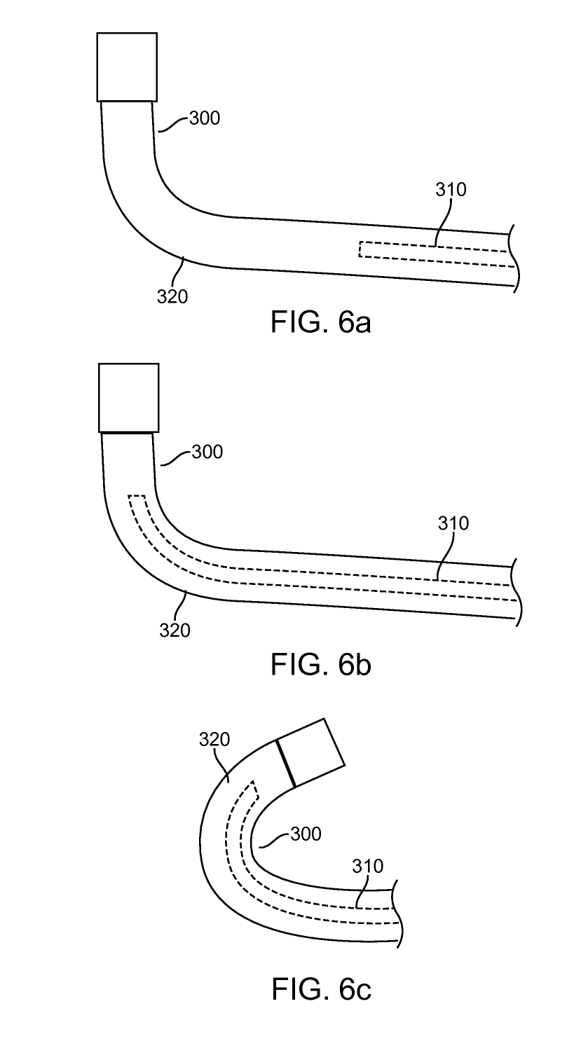

A catheter comprising an inner section 310 slidably positioned within an outer section 320 enables adjustable support by increasing the rigidity of at least a section of the catheter. Furthermore, a catheter comprising an inner section 310 slidably positioned within an outer section 320 is configured to affect, alter or reshape at least one curvature of at least one section of the catheter 300. As seen in FIG. 6a, a delivery catheter 300 comprising a curved distal section with an inner section 310 and an outer section 320. In one embodiment, the inner section 310 and outer section 320 are configured with different curvatures and/or constructed of materials with different rigidity, elasticity, and/or shape memory properties. As seen in FIG. 6a, the curvature of the distal section of the delivery catheter 300 is initially defined by the curvature of the outer section 310. As seen in FIG. 6a, the inner section 310 is placed initially proximal to the curved distal section of the inner section 310. As seen in FIG. 6b, when the inner section 310 is advanced towards the distal section, the inner section 310 affects the curvature configuration of the distal section of the delivery catheter 300. As seen in FIG. 6c, the curvature of the distal section of the delivery catheter 300 is reshaped or transformed to assume a new configuration as a result of the interactions between the inner section 310 and the outer section 320. The degree of transformation to the curvature of the distal section of the delivery catheter 300 may be calibrated by the position of the inner section 310 relative to the outer section 320, the initial curvature of the inner section 310, the difference between the curvature of the inner section 210 and the outer section 320, the rigidity, elasticity, and/or shape memory properties of the inner section 310 and the outer section 320, or a combination thereof. In addition to affect the curvature of the distal section of the delivery catheter 300, the interaction of the inner section 310 and the outer section 320 may be used to reinforce the pre-set curvature of the outer section 320 by fortifying the distal section of the outer section 320 to maintain the proper configuration when traversing a body region. Furthermore, the additional rigidity or support by the inner section 320 may prevent the catheter from prolapsing during operation.

It is noted that the embodiments of the delivery catheter 300 described and as shown in FIG. 5 an FIGS. 6a-6c may be used in combination with a bronchoscope 120. Such that the delivery catheter 300 may be adjusted to affect, maintain, transform, or amplify the curvature of the distal end of the bronchoscope 120. In one embodiment, the interactions between the bronchoscope 120, the inner section 310 of the delivery catheter 300, and the outer section 320 of the delivery catheter 300 creates greater flexibility to control the curvature or orientation of the bronchoscope 120. In one embodiment, the inner section 310 of the delivery catheter 120, the outer section 320 of the delivery catheter 300, and the bronchoscope 120 may be calibrated by altering the curvatures, the rigidity, elasticity, shape memory, and/or other material properties to achieve the desired curvature or orientation.

In the various embodiments described above, various sections of the delivery catheter and the bronchoscope may be constructed of the same material configured with different durometers. Alternatively, it is contemplated that various sections of the delivery catheter and the bronchoscope may be constructed of different materials of the same or different durometer. In one embodiment, the delivery catheter and the bronchoscope may be constructed of polyethylenes, nylon, nylon/pebax blends, flexible material, material with shape memory such as Nitinol, or a polymer or other materials and various combinations of thereof.

Also provided are methods for use the devices and systems. In one embodiment, at least one implantable device such as a bronchial isolation device is first inserted into the housing section 113 of the delivery catheter 110. In one embodiment, the distal section of the delivery catheter 110 is deployed into a bronchial passageway via the trachea such that the housing section 113 of the delivery catheter 110 is located at or near the target location in the bronchial passageway. Once the delivery catheter 110 and the attached bronchial isolation device is located at the target location, an operator can eject the bronchial isolation device from the housing section 113 into the bronchial passageway. In one embodiment, where the delivery catheter 110 comprise an inner section and an outer section as shown in FIG. 5, the curvature of the distal section may be adjusted by adjusting the position of the inner section relative to the outer section. In another embodiment, according to the transcopic method, the delivery catheter 110 is inserted into the working channel of the bronchoscope 120, which is deployed to the bronchial passageway either before or after the delivery catheter has been inserted into the bronchoscope 120. The position of the delivery catheter 110 may be adjusted once it is inserted into the bronchoscope 120 to affect the affect the curvature of a section of the bronchoscope 120.

Also provided are kits comprising one more devices described above. In certain embodiments, the kits at least include one delivery catheter. Kits may also include bronchoscope, and one or more implantable devices. Drugs and medication that can be injected into the cavity could also be included in the kit.

In addition to above-mentioned components, the subject kits typically further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

While the above is a complete description of various embodiments, any of a number of alternatives, modifications, and equivalents may be used in alternative embodiments. Therefore, the above description should not be taken as limiting the scope of the invention as it is defined by the appended claims.

What is claimed is:

1. A system for accessing a lung region, comprising:
a delivery catheter comprising a proximal section, a distal section, and a body disposed in-between, wherein the distal section comprises at least one curved section; and
a bronchoscope comprising a proximal section, a distal section, and a body disposed in-between, wherein the bronchoscope is configured to receive the delivery catheter;
wherein the delivery catheter is configured to affect a curvature of the distal section of the bronchoscope when the distal section of the delivery catheter is advanced at least partially through the distal end of the bronchoscope.

2. The system of claim 1, wherein at least a portion of the delivery catheter is constructed of a different material than the bronchoscope.

3. The system of claim 1, wherein the delivery catheter comprises an inner section and an outer section.

4. The system of claim 3, wherein the inner section and the outer section of the delivery catheter are configured to be slidably moveable relative to each other.

5. The system of claim 4, wherein at least a portion of the inner section and at least a portion of the outer section are constructed of different materials.

6. The system of claim 1, wherein the delivery catheter is configured to affect the curvature of the distal section of the bronchoscope by altering the degree of the curvature of the distal section of the bronchoscope.

7. The system of claim 1, wherein the delivery catheter is configured to affect the curvature of the distal section of the bronchoscope by fortifying the distal section of the bronchoscope.

8. A bronchial delivery catheter, comprising:
a proximal section;
a distal section with one or more curved sections;
a body disposed in-between the proximal section and a distal section; and
wherein the distal section comprises a housing section that is configured to house one or more bronchial implant.

9. The catheter of claim 1, wherein the delivery catheter comprises an inner section and an outer section.

10. The catheter of claim 9, wherein the inner section and the outer section are configured to be slidably moveable relative to each other.

11. The catheter of claim 10, wherein at least a portion of the inner section and at least a portion of the outer section are constructed of different materials.

12. The catheter of claim 1, wherein the delivery catheter is configured to be inserted into a bronchoscope, wherein the delivery catheter is configured to affect a curvature of a distal section of the bronchoscope by altering the degree of the curvature.

13. The catheter of claim 1, wherein the delivery catheter is configured to be inserted into a bronchoscope, wherein the delivery catheter is configured to fortify the distal section of the bronchoscope.
